(19) United States
(12) Patent Application Publication
(10) Pub. No.: US 2010/0228147 A1
(43) Pub. Date: Sep. 9, 2010

(54) PUNCTURING CYTODIAGNOSTIC DEVICE

(75) Inventor: Yuji Suda, Sendai-shi (JP)

Correspondence Address: KRATZ, QUINTOS & HANSON, LLP, 1420 K Street, N.W., 4th Floor, WASHINGTON, DC, 20005, US

(73) Assignee: SENDAI CITY MEDICAL CENTER, Miyagi (JP)

(21) Appl. No.: 12/279,900

(22) PCT Filed: Feb. 26, 2007
(86) PCT No.: PCT/JP2007/053498

§ 371 Date: Aug. 19, 2008

(30) Foreign Application Priority Data
Apr. 4, 2006 (JP) 2006-103150

(51) Int. Cl. A61B 10/02

(52) U.S. Cl. 600/567

(57) ABSTRACT

There is provided a device used for sampling a tissue within a lesion in a minimally invasive manner when a bronchus does not directly reach the lesion. A cell sampling needle 13 having a groove 16 engraved in a circumferential direction is formed at an end of a flexible wire 12. The wire and the needle are inserted into a flexible tube 11, and an end of the wire drawn out from a rear end of the tube is connected to a slider 29 inside an operation implement 20. The operation implement has a rack 22 and a pinion 27 engaging with the rack 22, and when the rack is pushed away via an operation bar 23, the pinion rotates, and in accordance with the motion, the slider first advances and then retreats. Accordingly, the cell sampling needle protrudes and withdraws in a moment from and into the end of the tube, thereby sampling of the lesion in the groove of the needle.

### TECHNICAL FIELD

The present invention relates to a puncturing cytodiagnostic device for sampling a cell of a lesion, which is used alone or in combination with an endoscope, an angiocatheter, or the like.

### BACKGROUND ART

When sampling a specimen from a lesion using an endoscope, it was conventionally conducted by extending forceps or a brush from an end of the endoscope (for example, refer to Patent Document 1). Particularly in peripheral lungs, most of lesions may exist away from the bronchus or outside a bronchial wall. In such a case, the brush is almost useless, and therefore the sampling must be conducted to dig out the lesion by using the forceps. As a result a surrounding tissue maybe destroyed, and it will be a real burden for patients. Furthermore, in case where the lesion cannot be dug out well, the sampling results in failure.

Patent Document 1: Japanese Patent Application Laid-open No. Hei 10-272089

### DISCLOSURE OF THE INVENTION

An object of the present invention is to sample a tissue within a lesion in a minimally invasive manner, in a case where the lesion is not directly visible.

A puncturing cytodiagnostic device of the present invention uses a cell sampling needle having a groove. The groove may be engraved around the needle in a spiral shape, or it may consist of a plurality of ring-shaped grooves with spaces therebetween. Such a needle is provided at an end of a flexible wire, and the wire and the needle are stored in a flexible tube. A rear end of the wire is drawn out from a rear end of the tube, and is connected to an operation implement. The wire is pushed and pulled with the use of the operation implement, so that the needle protrudes from and withdraws into an end of the tube.

This device is inserted into a working channel of an endoscope, an end of which is brought in close to the lesion, and is also directed thereto. Then, by operating the operation implement at hand, the cell sampling needle is protruded from and withdrawn into the end of the tube. When the needle stuck into the lesion is pulled out, the tissue of the lesion is sampled in the groove of the needle. With this device, even when a bronchus does not directly reach the lesion, the sampling of the cell of the lesion can be realized, so long as the end of the endoscope can reach near the lesion.

Instead of an endoscope, this device can be used by being inserted into an angiocatheter extended close to an organ having lesion, thereby sampling the lesion cell. Further, the device can also be applied for sampling a cell by being inserted into a flexible tube stuck from the surface of the body.

It is also applicable to design such that a hollow needle is extended at the end of the flexible tube, and the cell sampling needle is protruded from and withdrawn into an end of the hollow needle. If designed such that, it becomes possible to sample the lesion in the body by directly approaching thereto from the surface of the body without using the endoscope or the angiocatheter.

The operation implement could be simply formed by a ring which is connected at the rear end of the wire. However, in order to make the needle protrude from and withdraw into the end of the tube, such a ring-type operation implement is required two oposite operations: an operation for pushing a ring, and its subsequent operation for pulling the ring. To prevent the surrounding tissue from getting injured by the needle in a case where the endoscope or a patient moves, these two opposite operations should be instantaneously performed, but actually this is not easy.

Thus, the following type of an operation implement is preferably used. The operation implement has a slider supported in a housing thereof so that the slider can move in forward and rearward directions, a rear end of the wire being fixed to the slider. Also, a rack is supported in the housing in a forwardly and rearwardly movable manner, and a pinion engaging with the rack is rotatably supported in the housing. Furthermore, a convert means for converting a rotational movement of the pinion into forward and rearward movements of the slider is provided. Hence, when the rack is moved in a predetermined direction in a predetermined range, the slider advances and then retreats to its original position.

In this operation implement, the rack is pushed at one action from the retreat position to the advanced position, and the pinion engaged with the rack rotates. Then the convert means operates, and the slider advances first and then retreats to the original position. The movement of the slider is transmitted via the wire to the sampling needle at the end of the wire, and the sampling needle protrudes from the end of the tube and then withdraws at once. Since the needle can protrude and withdraw instantaneously, this implement minimizes the danger of meaninglessly hurting the surrounding tissue by the needle.

To convert the rotation of the pinion into a reciprocating movement of the slider, a crank mechanism may be used, but it requires a number of parts. So, the slider is provided with a slot which is positioned orthogonal to a movement direction of the slider, and an eccentric pin extending from the pinion which is engaged with the slot. By designing in this manner, a simple and compact structure can be obtained.

It is convenient if a protrusion amount of the needle from the end of the tube can be adjusted in accordance with a position and a size of a lesion. So it is preferable to provide a plurality of spaced apart fixing points on the slider for securing the end piece of the wire. In this structure, when the wire end is engaged with the frontward fixing point, the protrusion amount of the needle from the end of the tube becomes maximum, and when engaged with the rearward point it becomes minimum. By thus selecting fixing points, the protrusion amount of the needle can be adjusted.

In order to secure the safety, when the rack advances or retreats to its full extent, a stopper is preferable to work so that the rack does not move further. When the stopper works, the needle never protrudes. Therefore, an accident due to a wrong operation can be prevented.

### BRIEF DESCRIPTION OF DRAWINGS

FIG. 1 is an explanatory view of a main body of a cytodiagnostic device;

FIG. 2 is an explanatory view of a cell sampling needle;

FIG. 3 is a perspective view of an operation implement;

FIG. 4 is an elevational view of the operation implement in a state where a lid is removed;

FIG. 5A is an enlarged sectional view taken along a line A-A of FIG. 4;

FIG. 5B is an enlarged sectional view taken along a line B-B of FIG. 4;

FIG. 5C is an enlarged sectional view taken along a line C-C of FIG. 4;

FIG. 5D is an enlarged sectional view taken along a line D-D of FIG. 4;

FIG. 5E is an enlarged sectional view taken along a line E-E of FIG. 4;

FIG. 6 is a perspective view of a slider;

FIG. 7 is an explanatory view of a cytodiagnostic device being in use;

FIG. 8 shows a state where a lesioned cell is sampled in a peripheral lung; and

FIG. 9 is an explanatory view of a main body of a cytodiagnostic device showing another embodiment.

### BEST MODE FOR CARRYING OUT THE INVENTION

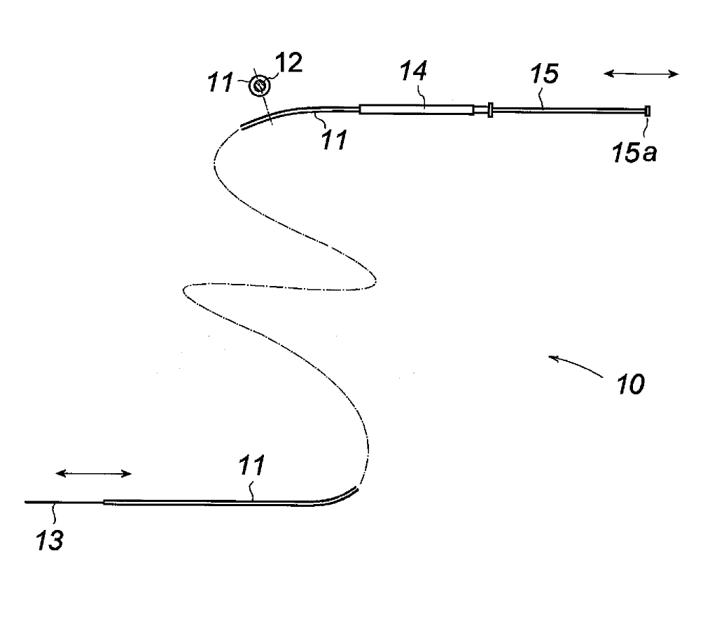

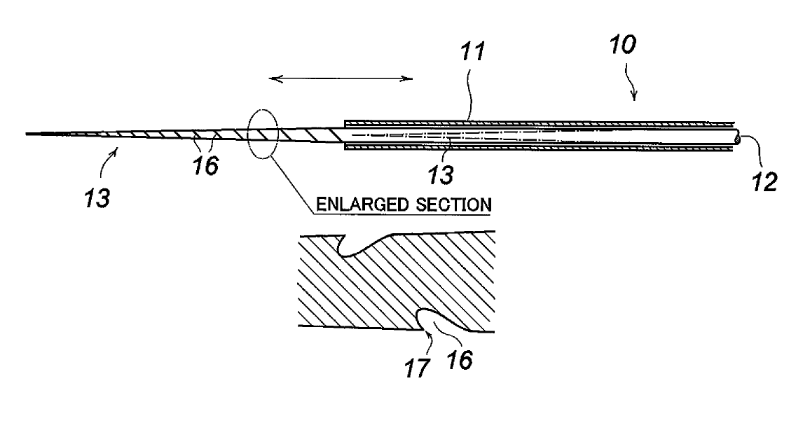

As shown in FIG. 1 and FIG. 2, a main body 10 of a puncturing cytodiagnostic device is structured to have a flexible wire 12 made of stainless or the like, being inserted into a flexible tube 11 made of polymeric material, in which a cell sampling needle 13 is provided at a end of the wire. This needle is also made to have an outside diameter capable of being stored in the tube 11. The needle 13 may be formed by manufacturing an end of the wire 12, or by bonding a needle which is prepared separately. The wire 12 is drawn out to the outside from an end piece 14 at a rear end of the tube, and an end piece 15 is attached to the pull-out end. The needle 13 is structured to protrude from and withdraw into the end of the tube 11 when the end piece 15 is pushed and pulled. In FIG. 2, the needle 13 in a state of being protruded and of being withdrawn are respectively shown by a solid line and a chain line. In the device, the tube 11 has an outside diameter of, for instance, about 1 mm, so that it can be inserted into a working channel of an extremely-fine bronchoscope. As shown in FIG. 2, the needle 13 has a tapered shape, and is provided with a spiral-shaped groove 16 at a circumferential surface thereof. The groove has cutting edge 17 at the fore side thereof to sample a tissue.

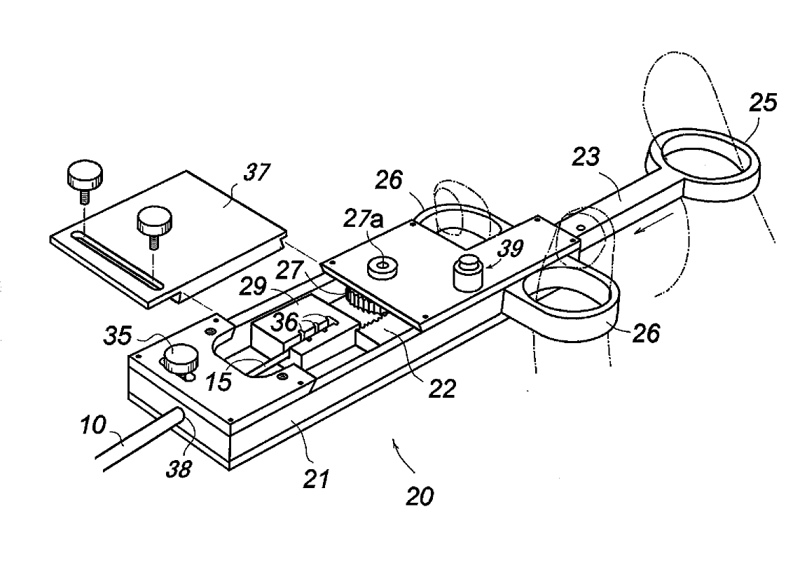

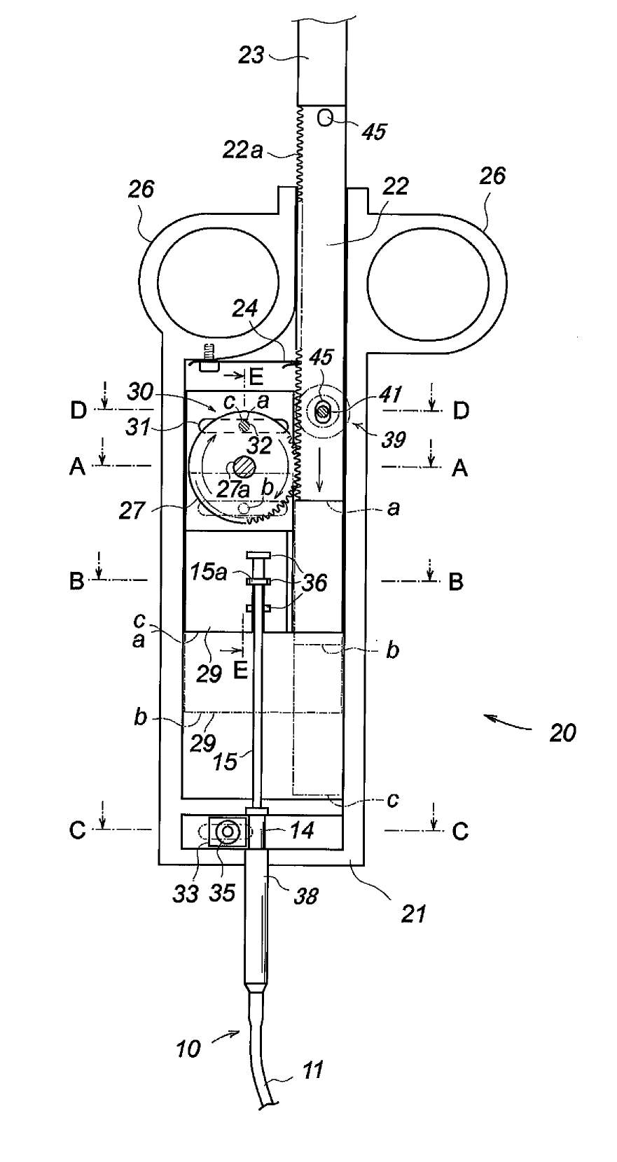

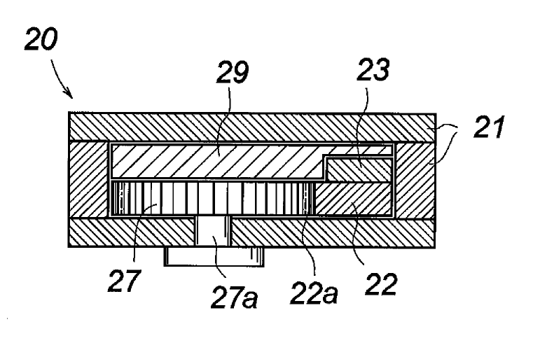

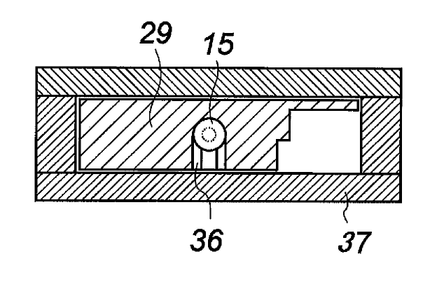

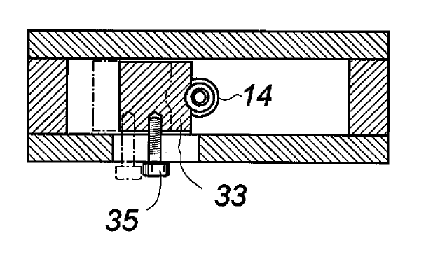

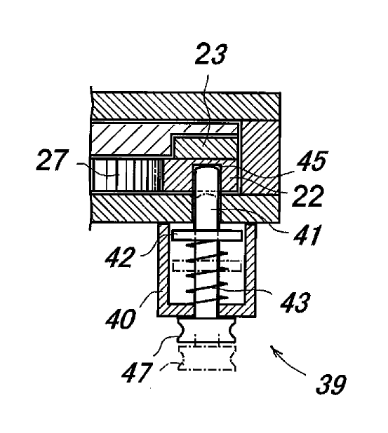

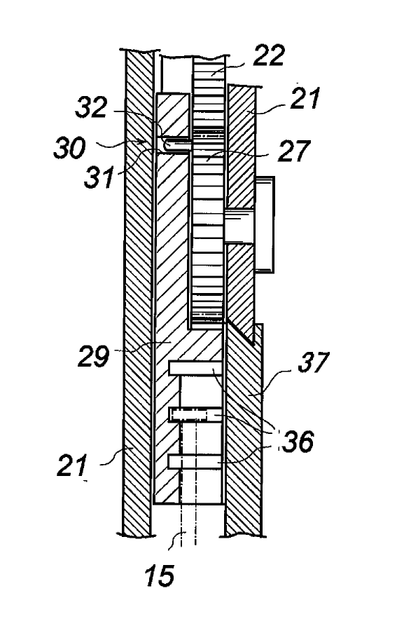

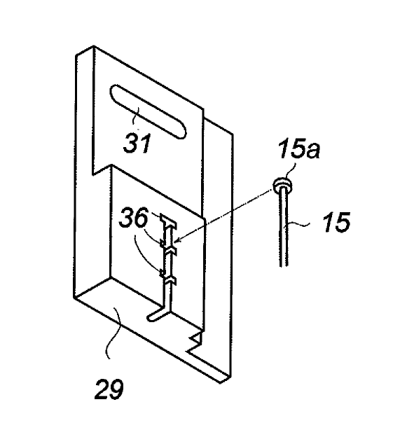

An operation implement 20 is used for pushing and pulling the wire 12 to make the cell sampling needle 13 protrude from and withdraw into the end of the tube 11, and a structure thereof will be described based on FIG. 3 io and FIG. 4. The operation implement has a box-shaped housing 21, and a rack 22 is supported therein in a forwardly and rearwardly (vertically, in FIG. 4) movable manner. An operation bar 23 extends to the outside from the rack 22 by penetrating the housing, and a ring 25 through which a thumb is inserted is provided at a rear end of the operation bar. Two rings 26 (for an is index finger and a middle finger) corresponding to the ring 25 are provided at a rear part of the housing. Inside the housing, a pinion 27 is rotatably supported on a supporting shaft 27a, and the pinion engages with gear teeth 22a of the rack 22 (shown in FIG. 5A). Inside the housing, a plate-shaped slider 29 is supported so as to be able to move in forward and rearward directions. Furthermore, a convert means 30 is provided, which converts a rotational movement of the pinion 27 into a reciprocating movement of the slider 29. The convert means 30 includes a slot 31 extending orthogonal to a movement direction of the slider 29 (as shown in FIG. 6), and an eccentric pin 32 projectingly provided on a rear side of the pinion 27, which engages with the slot (as shown in FIG. 5E).

At a front part of the housing of the operation implement, there is provided a stopper 33 for fixing the tube 11 of the puncturing cytodiagnostic device. The stopper can slide in a lateral direction inside the housing 21, and a neck portion of the end piece 14 of the tube inserted in the housing is held by this stopper and fixed by a screw 35 (as shown in FIG. 5C).

To connect the rear end of the wire 12 to the slider 29, it has three recesses 36 , as fixing points, which are aligned lengthwise and spaced. The head 15a of the end piece 15 can be fitted in one of recesses selectively (as shown in FIG. 6). For this connection, the housing 21 has a movable cover plate 37 as shown in FIG. 3.

When using the operation implement 20, the end piece 14 of the tube is inserted into the operation implement through a hole 38 provided at a front end of the operation implement, and the stopper 33 is applied to the neck portion of the end piece 14 and screwed down. Subsequently, the head 15a of the end piece of the wire drawn out from the tube is engaged with one of the three recesses 36 provided on the slider 29, and the movable cover plate 37 is put back on.

An operation of the operation implement proceeds such that a thumb is inserted through the ring 25, and the operation bar 23 at the retreat position is pushed forward at one action, as shown in FIG. 3. Then, as shown in FIG. 4, a front end of the rack 22 linked to the operation bar advances from the position indicated by letter “a” to the position “c”, the pinion 27 engaged with the rack rotates clockwise, and the slider is moved. In a first half process where the front end of the rack moves from “a” to “b” (“b” is a middle point between “a” and “c”), the eccentric pin 32 on the pinion advances from the position “a” to the position “b” while drawing an arc. At this time, the eccentric pin 32 pushes the slider 29 forward via the slot 31, so that the slider advances from the position “a” to the position “b”.

In a latter half process where the rack advances from “b” to “c”, the eccentric pin 32 retreats from the position “b” to the position “c” while drawing an arc, and during this time, the eccentric pin pulls the slider rearward, so that the slider goes back from “b” to “c” (“c” and “a” are the same position). In this embodiment, a stroke of the slider is about 15 mm. Thus, only by pushing the operation bar 23 forward, the slider 29 first advances and then retreats to the original position. The rear end of the wire 12 extending from the needle 13 is fixed to the slider, so that by pushing the operation bar 23 forward, the needle 13 first advances to protrude from the end of the tube 11, and then retreats to withdraw into the tube (as shown in FIG. 2).

The amount of the needle protrudes from the end of the tube (the protrusion amount) depends on which one of the three recesses 36 on the slider 29 is selected for fixing the end of the wire. In this embodiment, the three recesses 36 are lined up with 5 mm of spaces therebetween, and when the end of the wire is fixed to the frontward recess, the protrusion amount becomes maximum. When the wire is fixed to the middle recess, the protrusion amount lessens by 5 mm, and when fixed to the rearward recess, the protrusion amount lessens further by 5 mm. The numeral 24 indicates a leaf spring attached to the housing, a end of which engages with the gear teeth 22a of the rack so as to prevent the rack from moving freely unless an operating force is applied thereto (as shown in FIG. 4).

In order not to mistakenly protrude the needle from the end of the tube, it is designed such that, when the rack 22 is in the forward position indicated by letter “c” in FIG. 4, or in the backward position “a”, the stopper automatically functions, thereby preventing the rack 22 from moving. To explain this stopper 39 referring to FIG. 5D, a cap 40 is attached to the side of the housing, and a stopper shaft 41 is inserted so as to penetrate the cap. A collar 42 is fixed to the stopper shaft, and a coil spring 43 is compressedly provided between the collar and the cap. Thus, by the force of the spring, the stopper shaft 41 is urged into the housing. Meanwhile, the rack is provided with holes 45, into which the end of the stopper shaft 41 enters when the rack exists in the position “a” or “c” as shown in FIG. 4.

Before the usage, the rack is retreated and the stopper shaft 41 fits into the hole 45 as shown in FIG. 5D so as to prevent the needle from protruding by mistake. At the time of actual usage, as shown in the same drawing by a chain line, a head 47 is pinched with fingers, and pull out the shaft 41 from the hole 45 against the force of the spring 43. Under this state the operation bar 23 is pushed at a stroke, and the needle is protruded and then retreated. When the operation bar advances to its full extent, the stopper shaft automatically engages with the other hole 45, thereby locking the stopper.

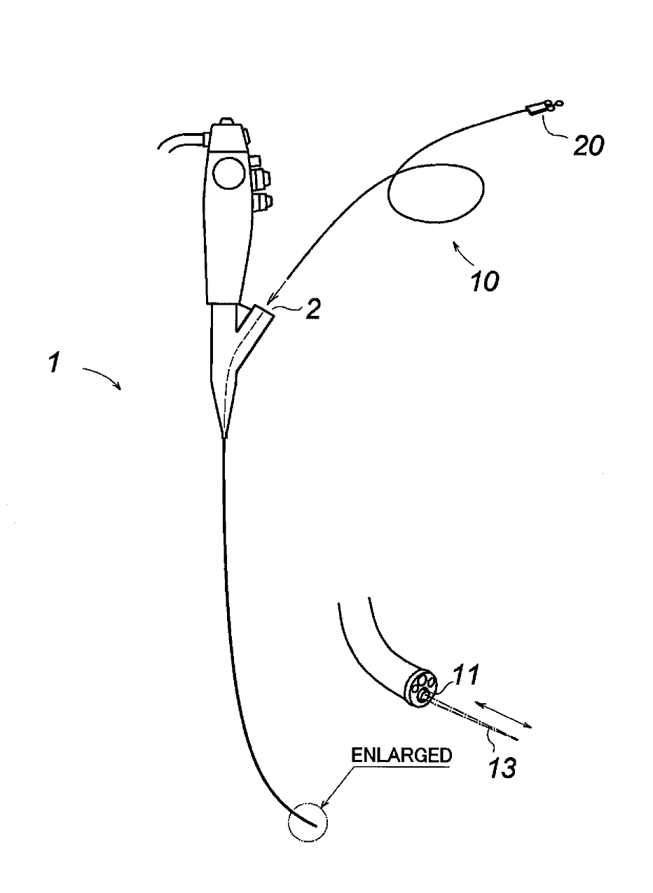

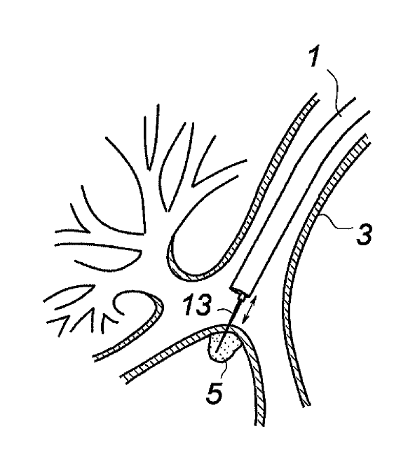

FIG. 7 shows a state where the cytodiagnostic device is used together with an endoscope. The main body 10 of the cytodiagnostic device is inserted into a working channel through an insertion port 2 of an endoscope 1, and is fixed at a position where the end of the tube 11 reaches an end of the endoscope. FIG. 8 shows a state where a sampling of a lesion is conducted. The endoscope 1 is inserted into a periphery of a bronchus 3 under X-ray fuluoroscopy, and an end of the endoscope is brought in close to a lesion 5 and is also directed thereto. Subsequently, by handling the operation implement 20, the lesion is punctured by the needle 13, to thereby sample a cell of the lesion.

The device is also suitably used for sampling a submucosal tumor by being inserted into a digestive endoscope. Conventionally, with respect to this type of lesion, there has been applied a complicated method in which an injection needle is extended from the end of the endoscope to be stuck into the lesion, and after sucking the lesion into a syringe barrel by pulling iₜs plunger of, the injection needle is withdrawn. With the use of this invented device, the sampling can be conducted in quite a short period of time with safety.

This device can also be inserted into, instead of the endoscope, an angiocatheter the end of which is extended close to an organ having a lesion, for sampling it. Further, it is also possible to directly insert a flexible tube from the surface of the body into a position in the vicinity of the lesion, and to insert the device of this invention into the flexible tube, to thereby conduct a sampling.

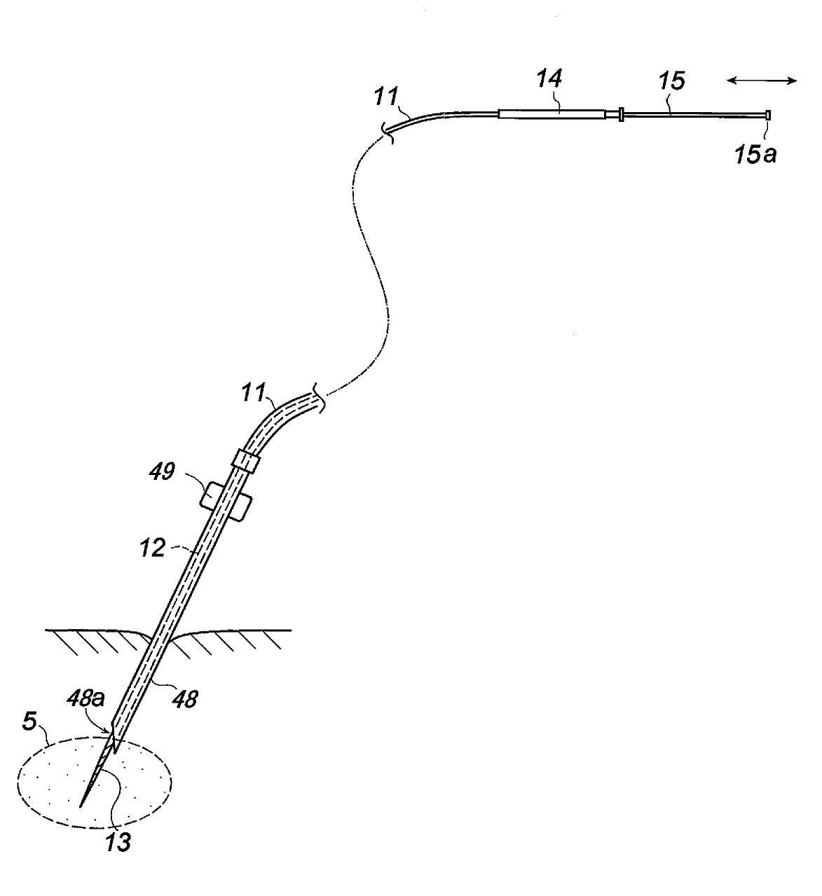

FIG. 9 shows another embodiment, in which a hollow needle 48 is extended at the end of the flexible tube 11, and the cell sampling needle 13 is protruded from and withdrawn into an end hole of the hollow needle. The hollow needle is made of metal or polymeric material, and an end thereof is diagonally tapered like an injection needle. A reference numeral 49 represents a handle. At the time of usage, the hollow needle is punctured from the skin into the lesion portion 5. A position of the hollow needle in the body can be confirmed by means such as ultrasonic wave, x-ray fluoroscopy, CT fluoroscopy, and CT guide. Subsequently, by handling the operation implement 20, the cell sampling needle 13 is protruded from and withdrawn into a end hole 48a of the hollow needle, to thereby conduct a sampling.

1. A puncturing cytodiagnostic device, comprising:
a flexible tube;
a flexible wire passing through said tube;
a cell sampling needle being provided at the end of said wire and having a groove at a circumferential surface thereof; and
an operation implement for pushing and pulling said wire to make said needle protrude from and withdraw into the end of said tube.

2. The puncturing cytodiagnostic device according to claim 1,
wherein a hollow needle is extended at the end of said flexible tube, and said cell sampling needle is protruded from and withdrawn into the end of the hollow needle.

3. The puncturing cytodiagnostic device according to claim 2,
wherein said operation implement, comprising:
a slider for securing said wire drawn out from a rear end of said tube, said slider being mounted in the housing of said operation implement for sliding movement in forward and rearward directions;
a rack being mounted in the housing for sliding movement in forward and rearward directions;
a pinion being rotatably supported in the housing and engaging with said rack; and
convert means for converting a rotational movement of said pinion into a forward and rearward movement of said slider so that said slider advances and then retreats to its original position when said rack is moved in a predetermined direction in a predetermined range.

4. The puncturing cytodiagnostic device according to claim 3,
wherein said convert means comprising:
a slot being provided on said slider and extending orthogonal to forward and rearward directions of said slider; and
an eccentric pin being provided to said pinion and engaging with said slot.

5. The puncturing cytodiagnostic device according to claim 3,
wherein said slider is provided with a plurality of fixing points spaced therebetween for fixing an end piece attached to a rear end of said wire

6. The puncturing cytodiagnostic device according to claim 3, further comprising
a stopper for locking said rack when said rack advances or retreats to its full extent.

7. The puncturing cytodiagnostic device according to claim 1,
wherein said operation implement, comprising:
a slider for securing said wire drawn out from a rear end of said tube, said slider being mounted in the housing of said operation implement for sliding movement in forward and rearward directions;
a rack being mounted in the housing for sliding movement in forward and rearward directions;
a pinion being rotatably supported in the housing and engaging with said rack; and
convert means for converting a rotational movement of said pinion into a forward and rearward movement of said slider so that said slider advances and then retreats to its original position when said rack is moved in a predetermined direction in a predetermined range.

8. The puncturing cytodiagnostic device according to claim 7,
wherein said convert means comprising:
a slot being provided on said slider and extending orthogonal to forward and rearward directions of said slider; and
an eccentric pin being provided to said pinion and engaging with said slot.

9. The puncturing cytodiagnostic device according to claim 7,
wherein said slider is provided with a plurality of fixing points spaced therebetween for fixing an end piece attached to a rear end of said wire

10. The puncturing cytodiagnostic device according to claim 7, further comprising
a stopper for locking said rack when said rack advances or retreats to its full extent.
